(19) United States
(12) Patent Application Publication
(10) Pub. No.: US 2005/0064512 A1
(43) Pub. Date: Mar. 24, 2005

(54) Emitter-binding peptides that produce a change in the spectral emission properties of the emitter

(76) Inventors: Michael Schirner, Berlin (DE); Kai Licha, Falkensee (DE); Andreas Menrad, Oranienburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/887,231

(22) Filed: Jul. 9, 2004

Related U.S. Application Data
(60) Provisional application No. 60/487,234, filed on Jul. 16, 2003.

(30) Foreign Application Priority Data
Jul. 9, 2003 (DE) 103 31 054.1

(51) Int. Cl. G01N 33/53; C07K 16/46

(52) U.S. Cl. 435/7.1; 530/391.1; 530/409

(57) ABSTRACT

This invention relates to emitter-binding peptides that produce a change in the spectral emission properties of the emitter in the case of an interaction of its antigen-binding pocket with the emitter. The emitter-binding peptides of the invention are in particular components of antibodies and antibody fragments.

This application claims the benefit of the filing date of U.S. Provisional Application Ser. No. 60/487,234 filed Jul. 16, 2003.

This invention relates to emitter-binding peptides that produce a change in the spectral emission properties of the emitter in the case of an interaction of its antigen-binding pocket with the emitter. The emitter-binding peptides of the invention are in particular components of antibodies and antibody fragments.

### BACKGROUND OF THE INVENTION

For the diagnostic detection of substances and determination of their concentration, now used in many cases are in-vitro diagnostic measuring processes that are based on biological molecules, such as, e.g., peptides, proteins, antibodies or oligonucleotides, which have a high affinity for a substance that is to be determined. For this purpose, proteins and peptides are preferably used, and antibodies and antibody fragments are especially preferably used.

Certain in-vitro diagnostic processes, such as, e.g., electrochemoluminescence, are based on the combination of various antibodies against the substance that is to be determined, whereby one antibody is used for the separation of the substance that is to be determined from the study sample, and the other antibody carries the diagnostically detected signal molecule. In the case of the diagnostic process of electrochemoluminescence, the labeled antibody is optically detected [Grayeski, M. L., Anal. Chem. 1987, 59, 1243].

In addition to the electroluminescence, the light-induced phosphorescence and the fluorescence can also be used as an optical property of molecules for diagnostic measuring processes. Compared to electroluminescence and phosphorescence, in particular fluorescence as an optical property of molecules offers the advantage of high detection sensitivity and high linearity of the measuring signal over a large dynamic range.

To detect the fluorescence of a fluorophore, various measuring processes were developed that use different principles within the fluorescence processes. Established measuring processes use, e.g., the weakening of polarized light (fluorescence polarization=FP), the measurement of the photon service life (fluorescence service life measurement —FLM), the bleaching properties (fluorescence photobleaching recovery—FPR) and the energy transfer between various fluorophores (fluorescence-resonance-energy transfer—FRET) [Williams, A. T., et al., Methods Immunol. Anal. 1993, 1, 466; Youn, H. J. et al., Anal. Biochem. 1995 Oswald, B. et al., Anal. Biochem. 2000, 280, 272; Szollosi, J. et al., Cytometry 1998, 34, 159].

Other detection processes are based on a change in the polarization plane or the detection of phosphorescence.

In the above-mentioned processes, the anti-substance antibodies that are used fulfill different purposes. On the one hand, they are used for separating the substance that is to be determined from the sample, but on the other hand, they also fulfill the object of locating or positioning different signal transmitters that are used on the substance that is to be examined. To detect, e.g., an antibody in a sample, primarily optical and radioactive measuring processes have been established, but also acoustic (see, e.g., Cooper, M. A. et al. Direct and Sensitive Detection of a Human Virus by Rupture Event Scanning. Nat Biotechnol. 2001 September; 19(9): 833-7) and magnetic measuring processes are known. The optical measuring processes have gained the maximum distribution [Nakamura, R. M., Dito, W. R., Tucker, E. S. (Eds.). Immunoassays: Clinical Laboratory Techniques for the 1980s. A. R. Liss, New York. Edwards, R. (ed.). Immunoassays: Essential Data, 1996, Wiley Europe].

In the majority of the already available measuring processes, the anti-substance antibody is labeled with a fluorophore. This labeling is carried out by specific and unspecific chemical coupling. The labeled antibody is added in excess to the study sample. This is necessary to bind all substance molecules that are to be examined. In addition, this process generally uses as a basis that one anti-substance antibody uses the separation of the substance that is to be examined and the second anti-substance antibody, which detects the study substance at another binding site, is labeled with a signaling molecule. In this way, a distortion of the measuring result by the unbonded, but signaling antibody can be avoided. This procedure, however, is associated with an elevated methodical and technical expense and higher costs, produced by the separation step. The high technical expense, which prevents establishing this process for high-speed diagnosis, has proven especially disadvantageous, however.

Antibodies and peptides that are directed against molecules of low molecular weight are already known. These also include antibodies and peptides against dye molecules. Simeonov, A. et al., in Science 2000, 290, 307-313 thus describe antibodies against stilbenes (“blue-fluorescent antibodies”). These antibodies catalyze specific photochemical isomerization processes and result in red-shifted absorption and fluorescence maxima in the UV-VIS spectral range (absorption shift maximum 12 nm, fluorescence shift 22 nm). Simeonov, A. et al., however, provide no reference whatsoever to a red shift while preserving the fluorescence quantum yield in the case of cyanine dyes in a wavelength range of 600-1200 nm.

Watt, R. M. et al. (Immunochemistry 1977, 14, 533-541) describe the spectral properties of the already known anti-fluorescein-antibody constructs. After binding the fluorescein, the antibody produces a shift of the absorption and fluorescence maximum in the visible spectral range, but only by 12 nm or 5 nm. In addition, a strong reduction of the fluorescence quantum yield (by about 90%) is carried out.

Rozinov, M. N. et al. (Chem. Biol. 1998, 5, 713-728) describe the selection of 12-mer peptides from phage libraries, which bind the dyes Texas Red, Rhodamine Red, Oregon Green 514 and fluorescein. For Texas Red, a red shift of the absorption and fluorescence was observed, but only by 2.8 nm or 1.4 nm.

In addition, antibodies against various dyes are already commercially available, e.g., against fluorescein, tetramethylrhodamine, Texas Red, Alexa fluorine 488, BODIPY FL, Lucifer Yellow and Cascade Blue, Oregon Green (Molecular Probes Company, Inc., USA). These are, however, polyclonal IgG antibodies for bioanalytical purposes, which have cross reactivities that are to some extent uncontrollable and are not produced from a strict selection process.

There is a further need for improved emitter-binding peptides and especially specific antibodies that are more suitable for the above-mentioned measuring processes. In this case, especially emitter-binding peptides that would produce a red shift while preserving the fluorescence quantum yield with cyanine dyes in the wavelength range of 600-1200 nm would be advantageous.

This object is achieved according to the invention by an emitter-binding peptide that is characterized in that the latter produces a change in the spectral emission properties of the emitter in the case of an interaction of its antigen binding pocket with the emitter, a process for the production of an emitter-binding peptide according to the invention, comprising the immunization of a suitable organism with an emitter, comprising a dye that is selected from the group of polymethine dyes, such as dicarbocyanine, tricarbocyanine, indotricarbocyanine, merocyanine, styryl, squarilium and oxonol dyes, and rhodamine dyes, phenoxazine or phenothiazine dyes and corresponding uses of an emitter-binding peptide, a nucleic acid, a host cell or an antibody or conjugate according to the invention as a diagnostic agent for in vitro diagnosis. Suitable embodiments are cited in the dependent claims.

A first aspect of this invention thus relates to an emitter-binding peptide, characterized in that the latter produces a change in spectral emission properties of the emitter in the case of an interaction of its antigen binding pocket with the emitter.

Preferred is an emitter-binding peptide according to the invention, whereby the emitter comprises a dye that exhibits at least an absorption maximum and/or fluorescence maximum within the spectral range of 700 to 1000 nm, preferably at least an absorption maximum and fluorescence maximum within the spectral range of 750 to 900 nm.

Further preferred is an emitter-binding peptide according to the invention, whereby the change in the emission properties of the part of the emitter is selected from a change in the polarization plane, the fluorescence intensity, the phosphorescence intensity, the fluorescence service life and a bathochromic shift of the absorption maximum and/or the fluorescence maximum. The invention is not limited to these special phenomena, however the term “change in the emission properties” within the scope of this invention is to comprise all physical phenomena or effects in which the high-energy radiation that occurs in the emitter is altered in its property and in this case this change is quantitatively dependent on the binding/non-binding of the substance-emitter conjugate or the substance-detecting agent-emitter-conjugate with its emitter-binding partner and the substance. In an embodiment, the substance is, for example, a peptide, protein, oligonucleotide and in particular an antibody or an antibody fragment. Within the scope of this invention, the antibody fragments are fragments that comprise at least the antigen-binding areas that contain the so-called “complementarity-determining regions” (“CDRs”). In this case, the antigen-binding areas most preferably comprise the complete variable chains VL and VH.

In an especially preferred aspect of the emitter-binding peptide according to this invention, the antibody or the antibody fragment is selected from polyclonal or monoclonal antibodies, humanized antibodies, Fab fragments, in particular monomeric Fab fragments, scFv fragments, synthetic and recombinant antibodies, scTCR chains and mixtures thereof.

Especially preferred here in the case of the synthetic and recombinant antibodies or antibody fragments are those from an HuCAL library (WO 97/08320; Knappik, (2000), J. Mol. Biol. 296, 57-86; Krebs et al. J Immunol Methods. 2001 Aug. 1; 254(1-2): 67-84). These can be present either as complete immunoglobulins or antibodies in one of the naturally occurring formats (IgA, IgD, IgE, IgG, IgM) or as antibody fragments, whereby the antibody fragments comprise at least the amino acid positions 4 to 103 for VL and 5 to 109 for VH, preferably the amino acid positions 3 to 107 for VL and 4 to 111 for VH and especially preferably the complete variable chains VL and VH (amino acid positions 1 to 109 for VL and 1 to 113 for VH) (numbering according to WO 97/08320).

In a preferred embodiment, the antibody or the antibody fragment comprises in this case at least one of the CDR areas contained in the sequences SEQ-ID Nos.: 1, 2, 5, 6, 9, 10, 13, 14, 17, 19, 21, 23, 25, 27, 29, 31, 33, 35, 37 and 39 (VL: CDR1 positions 24-34, CDR2 positions 50-56, CDR3 positions 89-96; VH: CDR1 positions 26-35, CDR2 positions 50-65, CDR3 positions 95-102), especially VL CDR3 or VH CDR3. Especially preferred in this case is an antibody that comprises one of the variable chains VL that are contained in the sequences SEQ-ID Nos.: 2, 6, 10, 14, 17, 19, 21, 23, 25, 27, 29, 31, 33, 35, 37 and 39 or a variable chain VL that is contained in one of the sequences SEQ-ID Nos.: 1, 5, 9 and 13 (or a fragment of such an antibody). Most preferred is an antibody that comprises a VH/VL pair that is contained in the following sequence pairs: SEQ-ID Nos.: 1+2; SEQ-ID Nos.: 5+6; SEQ-ID Nos.: 9+10; SEQ-ID Nos.: 13+14; SEQ-ID Nos.: 5+17; SEQ-ID Nos.: 5+19; SEQ-ID Nos.: 5+37; SEQ-ID Nos.: 9+21; SEQ-ID Nos.: 9+23; SEQ-ID Nos.: 9+25; SEQ-ID Nos.: 9+27; SEQ-ID Nos.: 9+29; SEQ-ID Nos.: 9+31; SEQ-ID Nos.: 9+33; SEQ-ID Nos.: 9+39; SEQ-ID Nos.: 13+35 (or a fragment of such an antibody). Especially preferred in this case are the Fab fragments MOR02628, MOR02965, MOR02977, MOR02969, MOR03263, MOR03325, MOR03285, MOR03201, MOR03267, MOR03268, MOR03292, MOR03294, MOR03295, MOR03309, MOR03293 and MOR03291. Starting from the thus described antibodies according to the invention, various possibilities to obtain new modified antibodies that also show the properties according to the invention follow in an obvious way for one skilled in the art.

It is known to one skilled in the art that especially the CDR areas both of the heavy chain and the light chain are responsible for the affinity as well as the selectivity and specificity. In this case, especially the CDR3 area of VH and the CDR3 area of VL play a role, followed by CDR2 of VH and CDR1 of VL, while CDR1 of VH and CDR2 of VL in most cases play a subordinate role. To optimize the affinity as well as the selectivity and specificity of the antibodies, therefore, in particular the CDR areas are suggested (see also, e.g., Schier et al., J. Mol. Biol. (1996) 263, 551). In this case, for example, one or more of the CDR areas can be exchanged, for example specifically for CDR areas of other antibodies that already show the properties according to the invention or else by libraries of corresponding CDR sequences (see for this purpose the optimization in Example 1), which either produce completely random variations or contain a more or less strong preference (tendency) in the direction of specific amino acids or combinations thereof. One skilled in the art is also able, moreover, to exchange complete variable chains in the same way for corresponding chains of other defined antibodies or for diverse libraries of such chains. In addition, processes to exchange one or more amino acid radicals in the CDRs specifically by mutagenesis are known to one skilled in the art. The identification of such changing amino acid radicals is carried out here, e.g., based on a comparison of the sequences of various antibodies and the identification of preserved or at least highly homologous radicals in the corresponding positions. In the changes to the CDRs, in this case one skilled in the art also has knowledge of so-called “canonical structures” (Al-Lazikani et al., J. Mol. Biol. (2000) 295, 979); Knappik et al. J. Mol. Biol. (2000) 296, 57), which have an influence on the three-dimensional arrangement of the CDR areas, and the optimization strategies corresponding to the design can be considered.

Moreover, techniques also to change the skeleton regions of antibodies or antibody fragments to obtain more stable or more expressible molecules are well known to one skilled in the art (WO 92/01787; Nieba et al. (1997) Protein Eng. 10, 435; Ewert et al. (2003) Biochemistry 42, 1517).

In addition to these already described strategies for modification of antibodies or antibody fragments, one skilled in the art, with knowledge of the antibodies according to the invention and the measuring processes that are described in this application, is also able to perform additional changes to the amino acid sequence or the composition of the described antibodies and to decide, by using the described assays and measuring process, whether modified antibodies have been produced whose properties match those that distinguish the antibodies according to the invention.

Another aspect of this invention relates to nucleic acid molecules that code one of the antibodies or an antibody fragment according to the invention. In a preferred embodiment, in this case these are nucleic acid molecules that code one of the variable chains VL that is contained in the sequences SEQ-ID Nos.: 2, 6, 10, 14, 17, 19, 21, 23, 25, 27, 29, 31, 33, 35, 37 and 39 or a variable chain VH that is contained in sequences SEQ-ID Nos.: 1, 5, 9 and 13. Especially preferred in this case are the sequences according to SEQ-ID Nos.: 3, 4, 7, 8, 11, 12, 15, 16, 18, 20, 22, 24, 26, 28, 30, 32, 34, 36, 38 and 40.

The emitter-binding peptide of this invention optimally exhibits a binding affinity of less than 50 nm and preferably of less than 10 nm.

Another aspect is an emitter-binding peptide of this invention, whereby the emitter comprises a dye that has at least an absorption maximum and/or fluorescence maximum within the spectral range of 700 to 1000 nm, preferably at least an absorption maximum and fluorescence maximum within the spectral range of 750 to 900 nm. The bathochromic shift of the dye is selected such that the shift of the absorption maximum and/or fluorescence maximum to higher wavelengths is carried out after interaction with the agent to detect the emitter by a value of greater than 15 nm, preferably greater than 25 nm and most preferably by approximately 30 nm. In this case, a shift does not necessarily have to be considered as such, which is a property of the dye. Usually, the shift would be measured as a change of an emission value matched to the die, i.e., at a certain singular wavelength. For this purpose, suitable optical agents for measurement that are known to one skilled in the art are provided. This also applies for the measurement of the change in the polarization plane, the fluorescence intensity, the phosphorescence intensity, the fluorescence service life and a bathochromic shift of the absorption maximum and/or the fluorescence maximum.

For the emitter-binding peptides according to the invention, it is preferred that the emitter that is used comprise a dye that is selected from the group of polymethine dyes, such as dicarbocyanine, tricarbocyanine, indotricarbocyanine, merocyanine, styryl, squarilium, and oxonol dyes, and rhodamine dyes, phenoxazine or phenothiazine dyes. In general, the emitter of the substance-emitter-conjugate according to the invention can comprise a cyanine dye of general formula (I) 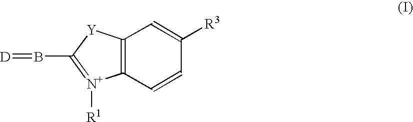
in which D stands for a radical (II) or (III) 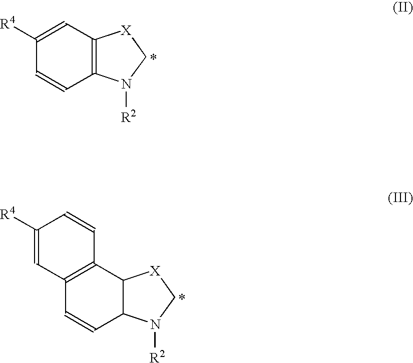
whereby the position that is labeled with the star means the point of linkage with radical B and can stand for the group (IV), (V), (VI), (VII) or (VIII) 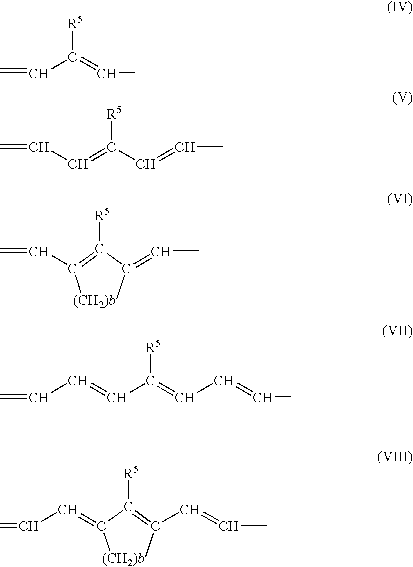
in which R¹ and R², independently of one another, represent a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or can be substituted with 0 to 5 hydroxy groups; R³ and R⁴, independently of one another, stand for the group —COOE¹, —CONE¹E², —NHCOE¹, —NHCONHE¹, —NE¹E², —OE¹, —OSO₃E¹, —SO₃E¹, —SO₂NHE¹ or -E¹ , whereby E¹ and E², independently of one another, represent a hydrogen atom, a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or is substituted with 0 to 5 hydroxy groups, R⁵ stands for a hydrogen atom, a methyl, ethyl or propyl group or a fluorine, chlorine, bromine or iodine atom, b means the number 2 or 3, and X and Y independently stand for O, S, ═C(CH₃)₂ or —(CH═CH)—, as well as salts and solvates of these compounds.

It was possible to find, surprisingly enough, that after highly affine binding of an antibody to a cyanine dye with absorption and fluorescence in the near-infrared spectral range (>750 nm), a shift of the absorption maximum and fluorescence maximum by about 30 nm to higher wavelengths was carried out (bathochromic shift). With use of this principle, it is thus possible, for example, via a large concentration range, to detect directly and spectrally separately a signal from a whole-blood sample, whereby the signal behaves linearly with respect to the concentration of the substance that is to be determined.

The emitter can form conjugates with substances. Within the scope of this invention, those of general formula
S-E
are used as substance-emitter conjugates, in which S stands for a substance that is to be examined and E stands for an emitter that comprises a part that reacts with a change in the emission properties in an interaction with the agents for detecting the emitter. As structural components of the conjugates according to the invention, i.a., dyes that have at least an absorption maximum and a fluorescence maximum within the spectral range of 600 to 1200 nm are suitable. In this case, dyes with at least an absorption maximum and a fluorescence maximum within the spectral range of 700 to 1000 nm are preferred. Dyes that meet these criteria are, for example, those of the following classes: polymethine dyes, such as dicarbocyanine, tricarbocyanine, merocyanine and oxonol dyes, rhodamine dyes, phenoxazine or phenothiazine dyes, tetrapyrrole dyes, especially benzoporphyrins, chlorines, bacteriochlorines, pheophorbides, bacteriopheophorbides, purpurines and phthalocyanines.

Preferred dyes are the cyanine dyes with absorption maxima between 750 and 900 nm, and with special advantage indotricarbocyanines. Structural components of the conjugates according to the invention are also the substances whose determination of concentration is to be carried out by means of the process according to the invention.

These are selected from, for example, antigens, such as proteins, peptides, nucleic acids, oligonucleotides, blood components, serum components, lipids, pharmaceutical agents and compounds of low molecular weight, especially sugars, dyes or other compounds with a molecular weight of under 500 Dalton.

Preferred dyes are the cyanine dyes with absorption maxima of between 750 and 900 nm, and with special advantage indotricarbocyanines.

The dyes contain structural elements, via which the covalent coupling to the substance structures is carried out. The latter are, e.g., linkers with carboxy groups, amino groups, and hydroxy groups.

In the case of an optical measurement, the latter can be carried out in a different way and is directed mainly according to the type of characteristic change in the spectral properties of the emitter (e.g., fluorophore). Generally preferred is a detection of the shift of the absorption wavelength and emission wavelength or the measurement of the absorption and/or fluorescence intensity at a wavelength that for the most part detects the portion of the emitter that is bonded to the antibody. Depending on the change in the spectral properties of the antibody-bonded fluorophore, other properties, such as, e.g., the photon service life, the polarization, and the bleaching behavior, can also be used for optical measurement.

The special advantage of fluorophores in the spectral range of near-infrared light lies in the low rate of shadowing by components of the blood. In this respect, deep penetration is made possible without the signal to be detected being relatively changed to any major extent.

Moreover, antibodies against fluorophores, which are able to change their spectral properties in the UV range after binding the fluorophore, are already known to one skilled in the art. By binding a fluorophore in the antigen binding pocket of an antibody, primarily the fluorescence intensity, the absorption maximum, the emission maximum, and the photon service life can be changed [Simeonov, A., et al., Science (2000) 307-313]. These known antibodies are directed against emitters (fluorophores), however, which have their absorption and fluorescence emission in the visible and UV range of the light.

Another aspect of this invention relates to the use of an emitter-binding peptide according to the invention for in vitro diagnosis.

For this purpose, the emitter-binding peptide according to the invention can also be present in a diagnostic kit, optionally together with other adjuvants. In addition, all of these kits according to the invention can contain special instructions and documents (e.g., calibration curves, directions for quantification, etc.).

The invention is now to be described in more detail below based on examples and the attached figures and sequences, without, however, being limited thereto. Here:

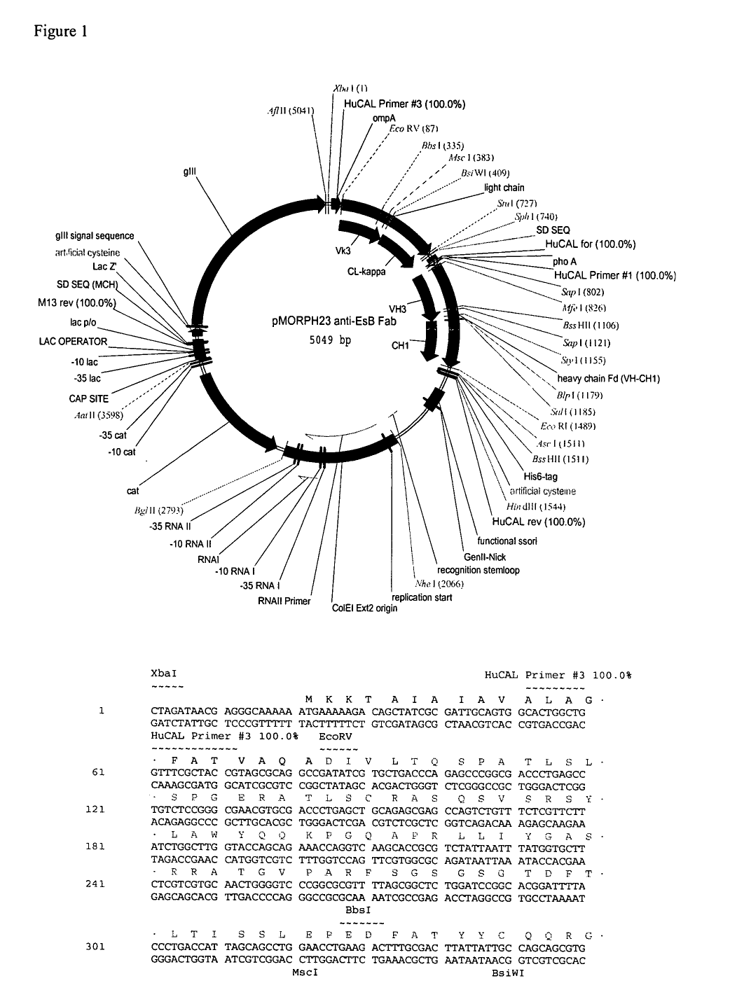

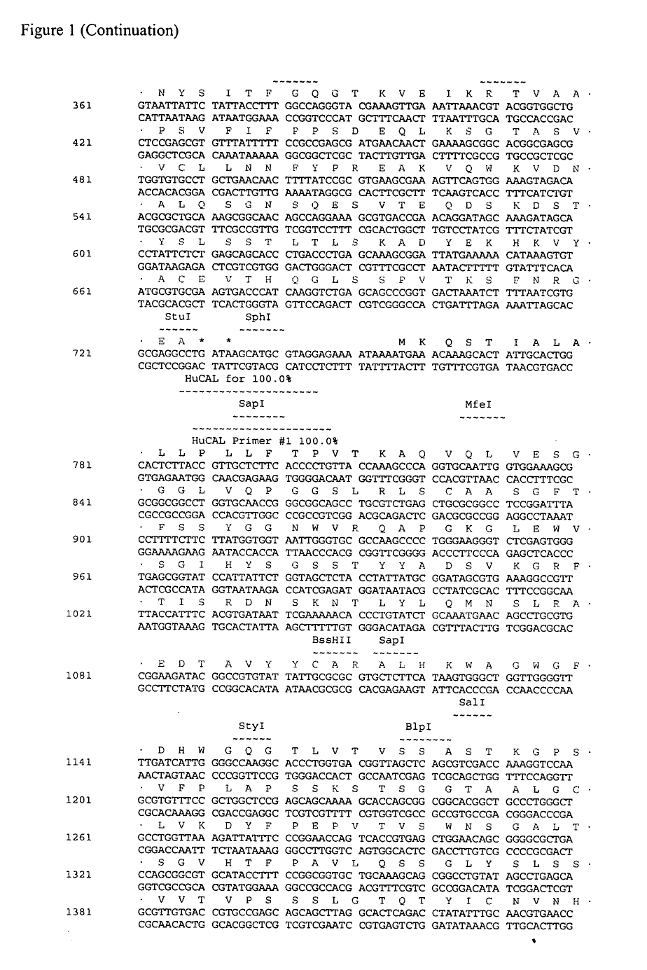

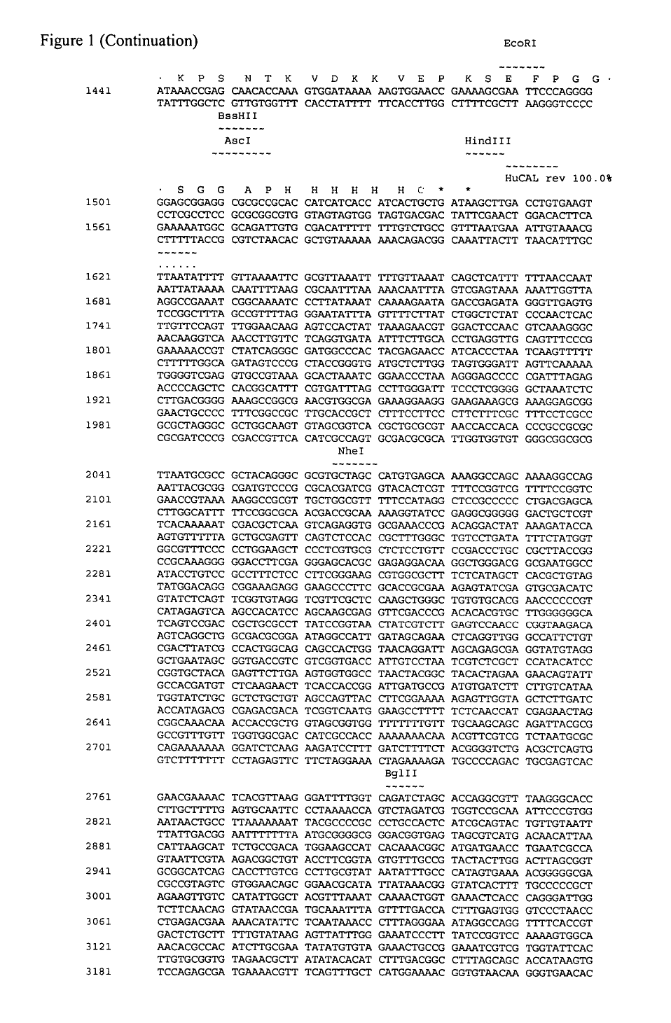

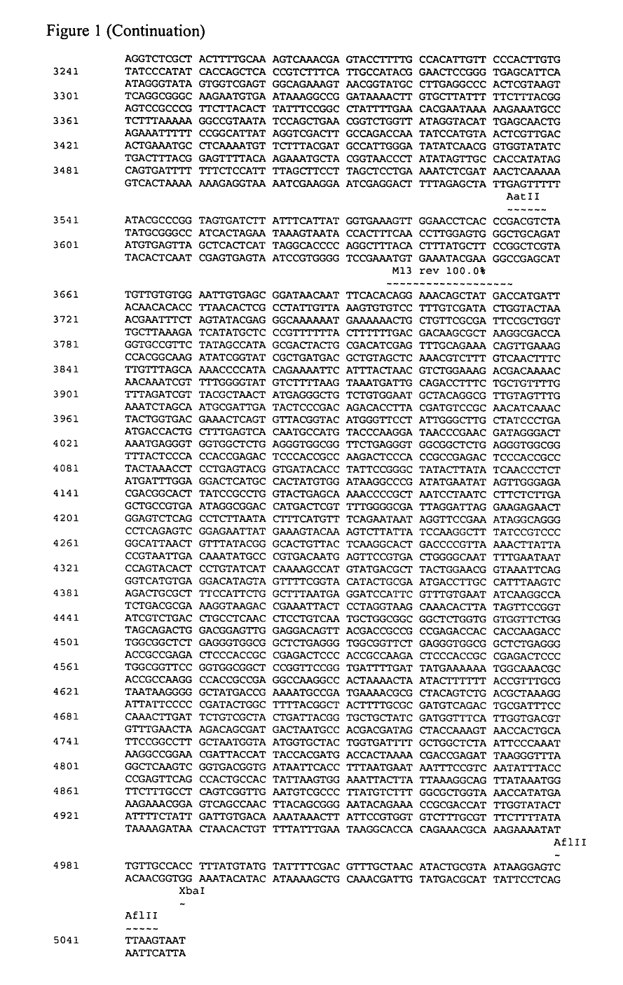

FIG. 1: The CysDisplay-Screening vector pMORPH23 (vector map and sequence),

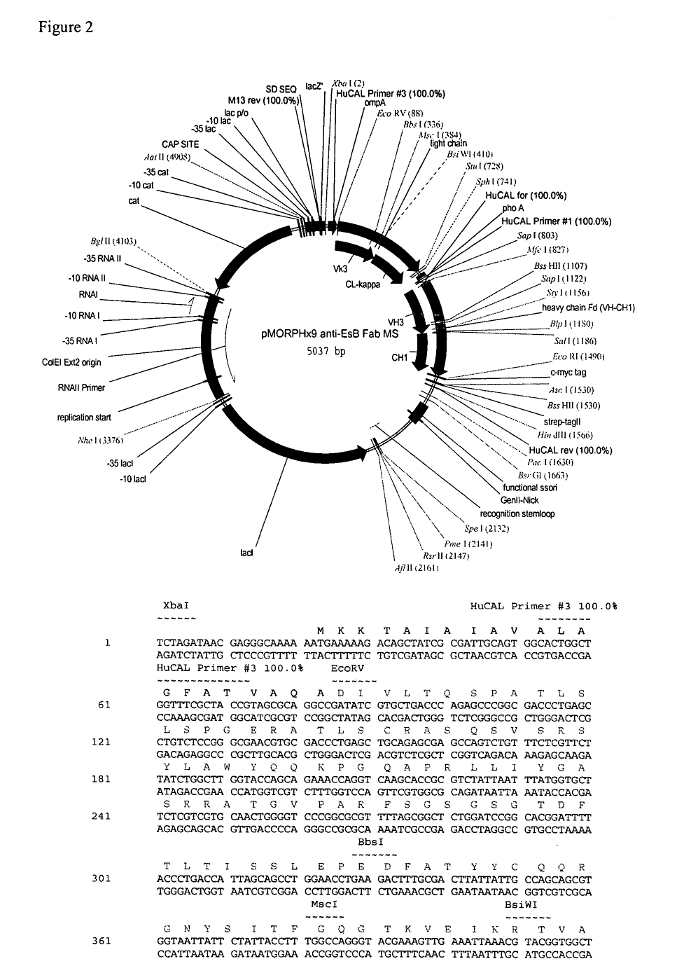

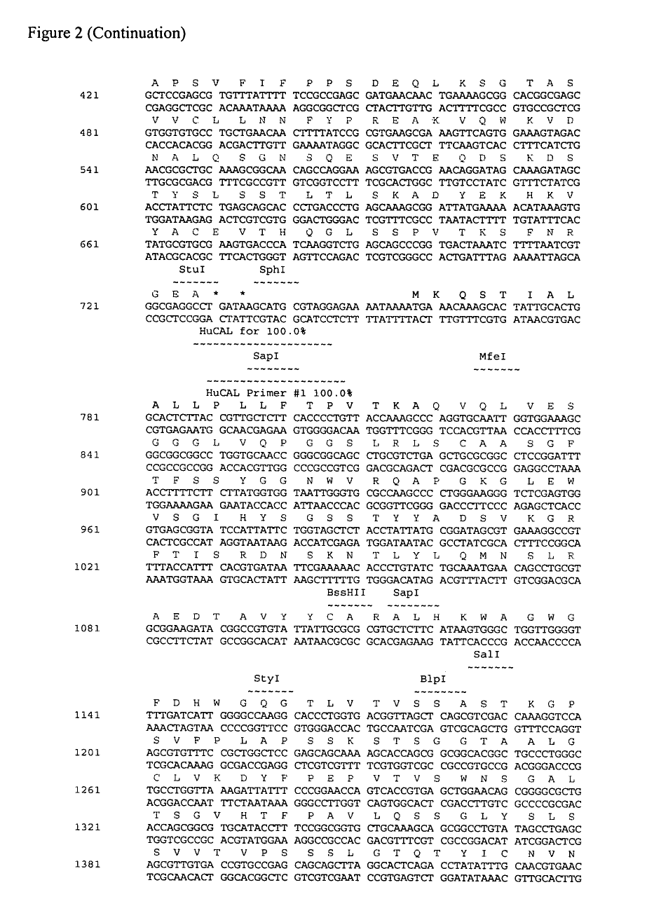

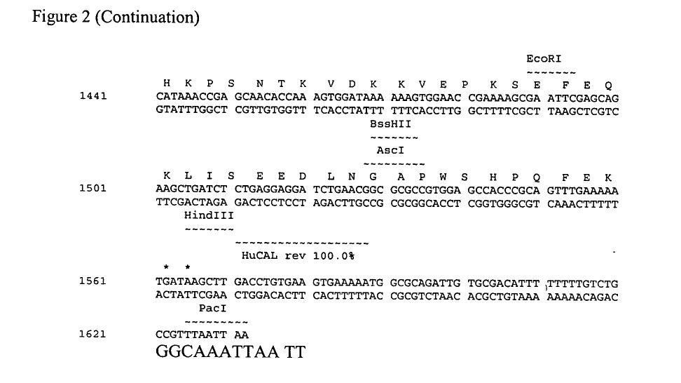

FIG. 2: The expression vector pMORPHX9 MS (vector map and sequence), and

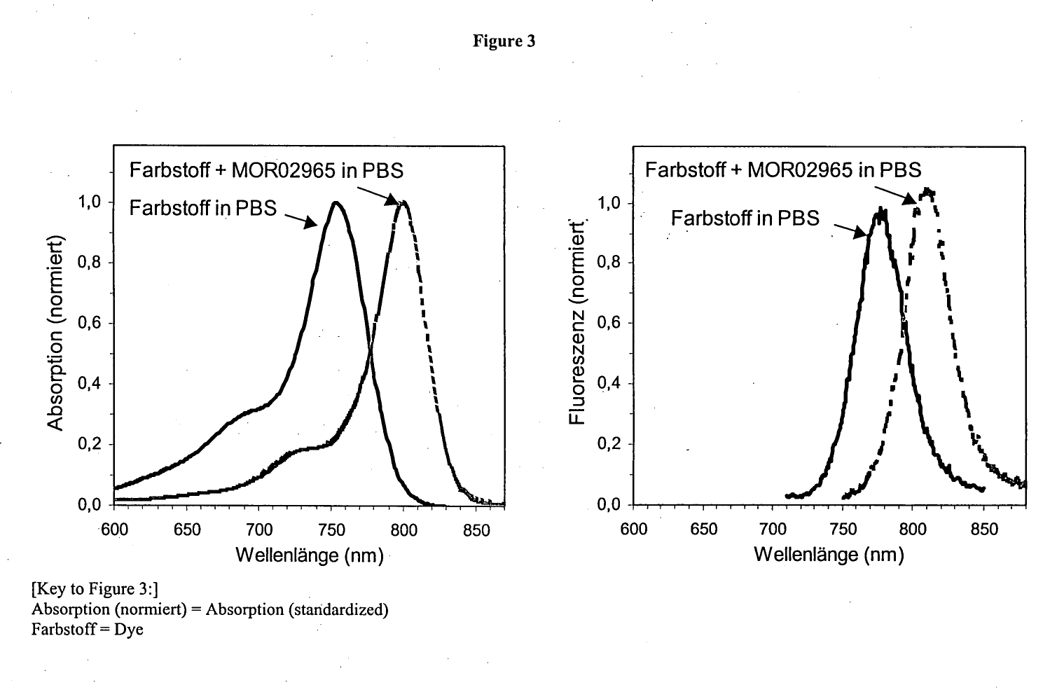

FIG. 3: Absorption spectrum (left) and fluorescence spectrum of the dye from Example 2 with and without the presence of antibody MOR02965 in PBS.

### EXAMPLES

### Example 1

### Selection, Production and Characterization of Emitter-Binding Antibodies: Selection of HuCAL GOLD Fab Antibody Fragments against the Cyanine Dye Fuji 6-4 (ZK203468) [Trisodium-3,3-dimethyl-2-{4-methyl-7-[3,3-dimethyl-5-sulfonato-1-(2-sulfonatoethyl)-3H-indolium-2-yl]hepta-2,4,6-trien-1-ylidene}-1-(2-sulfonatoethyl)-2,3-dihydro-1H-indole-5-sulfonate, Inner Salt]

HuCAL GOLD Antibody Library:

Antibody library HuCAL GOLD: HuCAL GOLD is a fully synthetic, modular human antibody library in the Fab antibody fragment format. HuCAL GOLD is based on the HuCAL-consensus-antibody genes that were described for the HuCAL-scFv1 library (WO 97/08320; Knappik, (2000), J. Mol. Biol. 296, 57-86; Krebs et al. J Immunol Methods. 2001 Aug. 1; 254(1-2): 67-84). In HuCAL GOLD, all six CDR areas are diversified by the use of so-called trinucleotide mutagenesis (Virnekäs et al. (1994) Nucleic Acids Res. 1994 Dec. 25; 22(25): 5600-7) corresponding to the composition of these areas in human antibodies, while in earlier HuCAL libraries (HuCAL-scFv1 and HuCAL-Fab1), only the CDR3-areas in VH and VL had been diversified corresponding to the natural composition (see Knappik et al., 2000). Moreover, a modified screening process, the so-called CysDisplay (WO 01/05950), is also found in HuCAL GOLD. Vector pMORPH23 that is used for the screening process is found in FIG. 1.

Vλ Positions 1 and 2.

The original HuCAL master genes were constructed with their authentic N-termini: VLλ1: QS (CAGAGC), VLλ2: QS (CAGAGC), and VLλ3: SY (AGCTAT). These sequences are found in WO 97/08320. In the production of the HuCAL-scFv1-library, these two amino acid radicals were changed in “DI” to facilitate the cloning (EcoRI site). These radicals were preserved in the production of HuCAL-Fab1 and HuCAL GOLD. All HuCAL libraries therefore contain VLλ genes with the EcoRV interface GATATC (DI) at the 5′-end. All HuCAL kappa genes (master genes and all genes in the libraries) in any case contain DI at the 5′-end, since these represent the authentic N-termini (WO 97/08320).

VH Position 1.

The original HuCAL-master genes were produced with their authentic N-termini: VH1 A, VH1B, VH2, VH4, and VH6 with Q(=CAG) as a first amino acid radical and VH3 as well as VH5 with E(=GAA). The corresponding sequences are found in WO 97/08320. In the cloning of HuCAL-Fab1 as well as the HuCAL GOLD library, the amino acid Q (CAG) was incorporated in all VH genes at this position 1.

Phagemid Production

Large amounts of phagemids were produced and concentrated by infection of E. coli TOP10F′ cells from the HuCAL GOLD antibody library or from the maturation libraries by means of helper phages. To this end, the HuCAL GOLD or the maturation libraries (in the TOP10F′ cells) were cultivated in 2×YT medium with 34 μg/ml of chloramphenicol/10 μg/ml of tetracycline/1% glucose at 37° C. up to an OD₆₀₀ of 0.5. Then, the infection was carried out with VCSM13 helper phages at 37° C. The infected cells were pelletized and resuspended in 2×YT/34 μg/ml of chloramphenicol/10 μg/ml of tetracycline/50 μg/ml of kanamycin/0.25 mmol of IPTG and cultivated overnight at 22° C. The phages were precipitated 2×0 with PEG from the supernatant and harvested by centrifuging (Ausubel (1998) Current Protocols in Molecular Biology. John Wiley Sons, Inc., New York, USA). The phages were resuspended in PBS/20% glycerol and stored at −80° C.

The phagemid amplification between the individual selection rounds was carried out as follows: log-phase E. coli TG1 cells were infected with selected phages and flattened out on LB-agar plates with 1% glucose/34 μg/ml of chloramphenicol. After incubation overnight, the bacteria colonies were scraped off, newly cultivated and infected with VCSM13 helper phages.

Primary Selection of Antibodies Against the Dye Fuji 6-4 (ZK203468)

The purified and concentrated phagemids of the HuCAL GOLD antibody library were used in a standard selection process. As antigens, BSA- or transferrin-coupled ZK203468 were used alternately. The antigens were taken up in PBS and applied at concentrations of 50 μg/ml on Maxisorp™ microtiter plates F96 (Nunc). The Maxisorp plates were incubated overnight at 4° C. (“coating”). After the Maxisorp plates were blocked with 5% milk powder in PBS, about 2E+13 HuCAL GOLD phages were added to the antigen-loaded, blocked-off wells and incubated there overnight or for two hours at room temperature. After several washing steps, which became more stringent with progressive selection rounds, bonded phages were eluted with 20 mmol of DTT or 100 μmol of unconjugated ZK203468. Altogether, three successive selection rounds were carried out, whereby the phage amplification was carried out between the selection rounds, as described above.

Sub-Cloning of Selected Fab Fragments for Expression

After the antibody selection that comprises three rounds, the Fab-coding inserts of the isolated HuCAL clones were subcloned in the expression vector pMORPHX9 _MS (see FIG. 2) to facilitate the subsequent expression of the Fab fragments. To this end, the purified plasmid-DNA of the selected HuCAL Fab clones was digested with the restriction enzymes XbaI and EcoRI. The Fab-coding insert was purified and ligated into the correspondingly digested vector pMORPHX9_MS. This cloning step results in the Fab-expressing vector pMORPHX9_Fab_MS. Fab fragments, which are expressed by this vector, carry two C-terminal tags (Myc tag and Strep tag II) for purification and detection.

Screening and Characterization of ZK203468-Binding Fab Fragments

Several thousand clones were isolated after the selection and sub-cloning and tested by means of ELISA in 384-well format for specific detection of the antigens ZK203468-BSA and -transferrin used in panning. Clones identified in this connection were studied in an inhibition-ELISA for efficient binding of the unconjugated dye.

This resulted in the parenteral Fab fragments MOR02628 (protein sequences SEQ-ID NO: 1 (VH-CH) and SEQ-ID NO: 2 (VL-CL); DNA sequences SEQ-ID NO: 3 (VH-CH) and SEQ-ID NO: 4 (VL-CL)), MOR02965 (protein sequences SEQ-ID NO: 5 (VH-CH) and SEQ-ID NO: 6 (VL-CL); DNA-sequences SEQ-ID NO: 7 (VH-CH) and SEQ-ID NO: 8 (VL-CL)), and MOR02977 (protein sequences SEQ-ID NO: 9 (VH-CH) and SEQ-ID NO: 10 (VL-CL); DNA sequences SEQ-ID NO: 1 (VH-CH) and SEQ-ID NO: 12 (VL-CL)), and MOR02969 (protein sequences SEQ-ID NO: 13 (VH-CH) and SEQ-ID NO: 14 (VL-CL); DNA sequences SEQ-ID NO: 15 (VH-CH) and SEQ-ID NO: 16 (VL-CL)), that bind efficiently to the non-conjugated dye ZK203468.

### Example 2

### Optimization of the Parenteral Antibody Fragments by Exchange of the LCDR3 Region

Cloning of the LCDR3 Libraries

The plasmid-DNA of the four parenteral clones MOR02628, MOR02965, MOR02969 and MOR02977 was digested with the restriction enzymes EcoRI and XbaI, and the complete Fab-insert from expression vector pMORPHX9_MS that was produced was subcloned in the correspondingly cut display vector pMORPH23. This step is necessary to prepare the geneIII, which is used in the presentation of the Fab-fragment on the phage surface. In another step, the four parenteral clones (now in pMORPH23) were digested with BpiI and SphI. In this connection, the LCDR3 region and the constant Clambda area were removed from the vector backbone. The corresponding vector-DNA fragment was isolated and purified. Parallel to this, the complementary BpiI/SphI fragment was isolated from the HuCAL-Fab 2 library, which contains a diversified LCDR3 region (with a variability from about 3E+8) plus constant Clambda area(=insert-DNA). Several μg of vector-DNA and compatible insert-DNA were ligated in a molar ratio of 1:2 with T4-DNA-ligase and transformed into electrocompetent TOP10F′ cells after a purification step. In this case, library sizes of 5E+8 to approximately 1E+9 clones were achieved per parenteral antibody.

The libraries on which clones MOR02628, MOR02969 and MOR02977 were based were combined (“Pool”). The MOR02965 library was treated separately (“Lead”). As already described, the corresponding phagemids were produced by infection with VCSM13 helper phages by means of these TOP10F′-maturation libraries.

Antibody Selection of Maxisorp™ Microtiter Plates

The purified and concentrated phagemids of the “lead” and “pool” libraries were used in a maturation-selection process under stringent conditions (long washing periods, displacement by purified, parenteral Fab proteins). As antigens, BSA- or transferrin-coupled ZK203468 were used alternately. These antigens were taken up in PBS and applied at low concentrations of 100-250 ng/ml on Maxisorp™ microtiter plates F96 (Nunc). The Maxisorp plates were incubated overnight at 4° C. (“coating”). After the Maxisorp plates were blocked with 5% milk powder in PBS, about 2E+13 HuCAL GOLD phages were added to the antigen-loaded, blocked-off wells and incubated there overnight or for two hours at room temperature. To increase the stringency, an additional 100 nm or 500 nm of purified Fab fragments of the parenteral clones were added during this incubation. After several extensive washing steps, bonded phages were eluted with 20 mmol of DTT. Altogether, two successive selection rounds were carried out, whereby the phage amplification was carried out between the selection rounds, as described above.

Antibody Selections on Neutavidin Strips

The purified and concentrated phagemids of the “lead” and “pool” libraries were used in addition in a second maturation-selection process under stringent conditions (long washing periods, displacement by purified, parenteral Fab proteins or free dye). As antigens, biotin-conjugated ZK203468 was used (alternately with alkyl or ether linkers). These antigens were taken up in PBS and mixed at low concentrations of 60 or 12 ng/ml with the approximately 2E+11 phages. The antigen-phage solutions were incubated overnight or for 2 hours at room temperature. To increase the stringency, an additional 0.5 μg/ml of purified Fab fragments of the parental clone or 40 nm/ml of ZK203468 was added during this incubation. The solutions that contain antigen-bonded phages were then applied to blocked neutravidin strips and incubated for 30 minutes to make possible the binding to the solid phase via the biotin radical of the antigen. After several washing steps, bonded phages were eluted with 20 mmol of DTT. Altogether, two successive selection rounds were carried out, whereby the phage amplification was carried out between the selection rounds, as described above.

Sub-Cloning of Selected Fab Fragments for Expression

After the selection (“maturation”), the Fab-coding inserts of the isolated HuCAL clones were subcloned in the expression vector pMORPHX9_MS to facilitate the subsequent expression. To this end, the purified plasmid-DNA of the selected HuCAL Fab clones was digested with the restriction enzymes XbaI and EcoRI. The Fab-coding insert was purified and ligated into the correspondingly digested vector pMORPHX9_MS. This cloning step results in the Fab-expressing vector pMORPHX9_Fab_MS. Fab fragments, which are expressed by this vector, carry two C-terminal tags (Myc tag and Strep tag II) for purification and detection.

Identification of Optimized Antibody Fragments

To identify antibodies with improved affinities for the dye Fuji 6-4, the clones were isolated from the selections and screened in ELISAs in 384-well format. To this end, ZK203468-BSA was applied on the ELISA-microtiter plates. The Fab fragments that are to be examined were added as non-purified bacterial lysates. To study in addition to the binding to the antigen conjugate the binding to the free dye, identical screening plates with bacterial lysate and additional free dye were mixed in two different concentrations. The inhibition of the Fab binding to the solid phase that resulted in this case owing to the unconjugated dye indicated antibodies that do not specifically detect the dye conjugate but rather only the free dye. In this connection, isolated clones were characterized exactly in solution-inhibition tests in the ELISA format and the Luminex device, and their affinities for Fuji 6-4 were determined.

The following clones showed improved affinities in comparison to the parenteral antibodies:

| Name | Parenteral Fab | LCDR3-Sequence |
|---|---|---|
| MOR02969 | — | SSYTYRVGGM |
| MOR03291 | MOR02969 | ASYDYKSKNI |
| MOR02965 | — | SSWDSSFSW |
| MOR03263 | MOR02965 | SSWDVSLEW |
| MOR02977 | — | QSWTTRPLNR |
| MOR03201 | MOR02977 | SSWTSYFHIR |
| MOR03267 | MOR02977 | QAWDSNFKNR |
| MOR03292 | MOR02977 | QSWAPLFKMR |
| MOR03295 | MOR02977 | QSWDSALSNR |

In summary, the affinity of the parenteral MOR02977 in comparison to MOR03267 could be improved by the factor 140. All other identified clones showed improvements by factors 2-70 in comparison to the respective parenteral Fab.

### Example 3

### Photophysical Characterization of Dye-Antibody Complexes and Determination of Spectral Shifts/Fluorescence Quantum Yields

Dye-antibody complexes based on antibodies with binding to the indotricarbocyanine dye trisodium-3,3-dimethyl-2-{4-methyl-7-[3,3-dimethyl-5-sulfonato-1-(2-sulfonatoethyl)-3H-indolium-2-yl]hepta-2,4,6-trien-1-ylidene}- 1-(2-sulfonatoethyl)-2,3-dihydro-1H-indole-5-sulfonate, inner salt, were examined (see Examples 1 and 2). Solutions of the concentration of 1 μmol/l of the above-mentioned dye and 2.4 μmol/l of the respective antibody in PBS were produced and incubated for 2 hours at room temperature. The absorption maxima were determined with a spectral photometer (Perkin-Elmer, Lambda2). The fluorescence maxima and fluorescence quantum yields were determined with a SPEX fluorolog (wavelength-dependent sensitivity calibrated by lamp and detector) relative to indocyanine green (Q=0.13 in DMSO, J Chem Eng Data 1977, 22, 379, Bioconjugate Chem 2001, 12, 44). From the absorption and fluorescence maxima, the spectral shifts were calculated relative to the maxima of a solution of the above-mentioned dye without antibodies in PBS (1 μmol/l) (absorption max. 754 nm, fluorescence max. 783 nm, fluorescence quantum yield 10%).

| | | | | | | |
|---|---|---|---|---|---|---|
| | | | SEQ-ID | SEQ-ID | SEQ-ID | SEQ-ID |
| | Parenteral | LCDR3- | NO. VH | NO. VL | NO. VH | NO. VL |
| Name | Fab | Sequence | Protein | Protein | DNA | DNA |

| | | | | | | |
|---|---|---|---|---|---|---|
| | MOR02628 | AAWDFRKRLN | 1 | 2 | 3 | 4 |
| | MOR02965 | SSWDSSFSW | 5 | 6 | 7 | 8 |
| | MOR02977 | QSWTTRPLNR | 9 | 10 | 11 | 12 |
| | MOR02969 | SSYTYRVGGM | 13 | 14 | 15 | 16 |
| MOR02965 | — | SSWDSSFSW | 5 | 6 | 7 | 8 |
| MOR03263 | MOR02965 | SSWDVSLEW | 5 | 17 | 7 | 18 |
| MOR03325 | MOR02965 | ASWDKSLQW | 5 | 19 | 7 | 20 |
| MOR03285 | MOR02965 | QAWTGSYAT | 5 | 37 | 7 | 38 |
| MOR02977 | — | QSWTTRPLNR | 9 | 10 | 11 | 12 |
| MOR03201 | MOR02977 | SSWTSYFHIR | 9 | 21 | 11 | 22 |
| MOR03267 | MOR02977 | QAWDSNFKNR | 9 | 23 | 11 | 24 |
| MOR03268 | MOR02977 | RSWDSNLSYS | 9 | 25 | 11 | 26 |
| MOR03292 | MOR02977 | QSWAPLFKMR | 9 | 27 | 11 | 28 |
| MOR03294 | MOR02977 | QTWTSSFSSR | 9 | 29 | 11 | 30 |
| MOR03295 | MOR02977 | QSWDSALSNR | 9 | 31 | 11 | 32 |
| MOR03309 | MOR02977 | QTWDHGFTHR | 9 | 33 | 11 | 34 |
| MOR03293 | MOR02977 | SSWTTIYRNR | 9 | 39 | 11 | 40 |
| MOR02969 | — | SSYTYRVGGM | 13 | 14 | 15 | 16 |
| MOR03291 | MOR02969 | ASYDYKSKNI | 13 | 35 | 15 | 36 |

The results are summarized in the following table:

| | | Absorption | Fluorescence | Absorption | Fluorescence | Fluorescence |
|---|---|---|---|---|---|---|
| | Parenteral | Maximum | Maximum | Shift | Shift | Quantum |
| Antibody | Antibody | (nm) | (nm) | (nm) | (nm) | Yield (%) |
| Free Dye | — | 754 | 783 | — | — | 10.0 |
| MOR02965 | — | 799 | 815 | 45 | 32 | 13.0 |
| MOR03263 | MOR02965 | 798 | 810 | 44 | 27 | 10.0 |
| MOR03325 | MOR02965 | 803 | 819 | 49 | 36 | 18.0 |
| MOR02977 | — | 773 | 788 | 19 | 5 | 24.5 |
| MOR03201 | MOR02977 | 786 | 804 | 32 | 21 | 21.0 |
| MOR03267 | MOR02977 | 783 | 802 | 29 | 19 | 38.5 |
| MOR03268 | MOR02977 | 786 | 805 | 32 | 22 | 30.0 |
| MOR03292 | MOR02977 | 781 | 800 | 27 | 17 | 25.0 |
| MOR03294 | MOR02977 | 783 | 803 | 29 | 20 | 22.0 |
| MOR03295 | MOR02977 | 784 | 803 | 30 | 20 | 23.0 |
| MOR03309 | MOR02977 | 784 | 803 | 30 | 20 | 21.5 |

### Example 4

### Construction of Expression Vectors for the Expression of HuCAL Immunoglobulins

Cloning of the heavy chain: the “multiple cloning site” of the vector pCDNA3.1+ (Invitrogen) is removed (NheI/ApaI), and a placeholder that is compatible with the restriction interfaces of the HuCAL design is used for the ligation of the leader sequence (NheI/EcoRI), the VH domains of the Fab fragment (MunI/), and the constant immunoglobulin regions (BlpI/ApaI). The leader sequence (EMBL 83133) is equipped with a contact sequence (Kozak, 1987). The constant regions of human IgG (PIR J00228), IgG4 (EMBL K01316), and serum-IgA1 (EMBL J00220) are divided up into overlapping oligonucleotides with a length of, for example, 70 bases. “Silent mutations” are introduced to remove the reaction interfaces that are not compatible with the HuCAL design. The oligonucleotides are linked by “overlap extension-PCR”.

During the subcloning of the Fab fragments in an IgG molecule, the heavy chain of the Fab fragment is cut out via MfeI/BlpI and ligated into the vector, which is opened with EcoRI/BlpI. EcoRI (g/aattc) and MfeI (c/aattg) have two compatible cohesive ends (aatt), and the sequence of the original MfeI interface in the Fab fragments is changed from: c/aattg to g/aattg to the ligation in the IgG expression vector, by which, on the one hand, both the MfeI interface and the EcoRI interface are destroyed, and, on the other hand, an amino acid exchange from Q (codon: caa) to E (codon: gaa) takes place.

Cloning of the light chain. The “multiple cloning site” of pCDNA3.1/Zeo+ (Invitrogen) is replaced by two different placeholders. The k-placeholder contains restriction interfaces for the incorporation of a k-leader sequence (NheI/EcoRV), the HuCAL Fab Vk domains (EcoRV/BsiWI), and the constant region of the k-chain (BsiWI/ApaI). The corresponding interfaces in the 1-placeholder are NheI/EcoRV (1-leader), EcoRV/HpaI (V1-domains), and HpaI/ApaI (constant region 1-chain). The k-leader (EMBL Z00022) as well as the 1-leader (EMBL J00241) are both provided with Kozak sequences. The constant regions of human k-(EMBL L00241) and 1-chains (EMBL M18645) are both assembled by “overlap extension-PCR,” as described above.

Generation of IgG-expressing CHO cells. CHO-K1 cells are co-transfixed with an equimolar mixture of expression vectors for the heavy and light IgG chains. Doubly resistant transfectants are selected with 600 mg/ml of G418 and 300 mg/ml of Zeocin (Invitrogen) followed by limited dilution. The supernatant of individual clones is checked for IgG expression by “capture-ELISA.” Positive clones are cultured in RPMI-1640 medium, which is provided with 10% “ultra-low IgG-FCS” (Life Technologies). After the pH of the supernatant is set at 8.0 and after sterile filtration, the solution is subjected to a standard protein A-column chromatography (Poros 20 A, PE Biosystems).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure[s] of all applications, patents and publications, cited herein and of corresponding German application No. 103 31 054.1, filed Jul. 9, 2003, and U.S. Provisional Application Ser. No. 60/487,234, filed Jul. 16, 2003 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Emitter-binding peptide, characterized in that the latter produces a change in the spectral emission properties of the emitter in the case of an interaction of its antigen binding pocket with the emitter.

2. Emitter-binding peptide according to claim 1, whereby the emitter comprises a dye that has at least an absorption maximum and/or fluorescence maximum within the spectral range of 700 to 1000 nm, preferably at least an absorption maximum and fluorescence maximum within the spectral range of 750 to 900 nm.

3. Emitter-binding peptide according to claim 1 2, which is selected from antibodies or antibody fragments, such as, for example, Fab fragments, scFv fragments, scTCR chains, single-chain antibodies and mixtures thereof.

4. Emitter-binding peptide according to claim 3, comprising one of the VH/VL pairs, which is contained in one of the following sequence pairs: SEQ-ID Nos.: 1+2; SEQ-ID Nos.: 5+6; SEQ-ID Nos.: 9+10; SEQ-ID Nos.: 13+14; SEQ-ID Nos.: 5+17; SEQ-ID Nos.: 5+19; SEQ-ID Nos.: 5+37; SEQ-ID Nos.: 9+21; SEQ-ID Nos.: 9+23; SEQ-ID Nos.: 9+25; SEQ-ID Nos.: 9+27; SEQ-ID Nos.: 9+29; SEQ-ID Nos.: 9+31; SEQ-ID Nos.: 9+33; SEQ-ID Nos.: 9+39; and SEQ-ID Nos.: 13+35.

5. Emitter-binding peptide according to claim 1, whose binding affinity for the emitter is less than 50 nm and preferably less than 10 nm.

6. Emitter-binding peptide according to claim 1, whereby the change in the emission properties of the emitter is selected from a change in the polarization plane, the fluorescence intensity, phosphorescence, especially phosphorescence intensity, service life of the fluorescence and a bathochromic shift of the absorption maximum and/or fluorescence maximum.

7. Emitter-binding peptide according to claim 1, whereby the shift of the absorption and/or fluorescence maximum to higher wavelengths after interaction with the agent to detect the emitter is carried out by a value of greater than 15 nm, preferably greater than 25 nm, and most preferably by approximately 30 nm.

8. Emitter-binding peptide according to claim 2, whereby the dye is selected from the group of polymethine dyes, such as dicarbocyanine, tricarbocyanine, indotricarbocyanine, merocyanine, styryl, squarilium and oxonol dyes and rhodamine dyes, phenoxazine or phenothiazine dyes.

9. Emitter-binding peptide according to claim 2, whereby the dye comprises a cyanine dye of general formula (I) 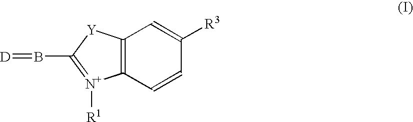
in which D stands for a radical (II) or (III) 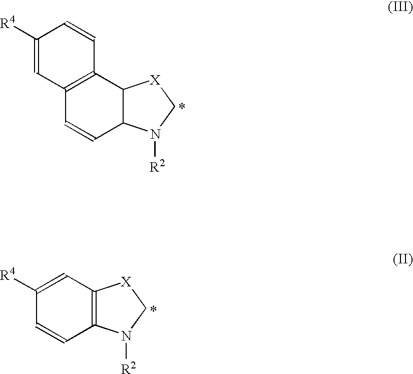
whereby the position that is labeled with the star means the point of linkage with radical B and can stand for the group (IV), (V), (VI), (VII) or (VIII) 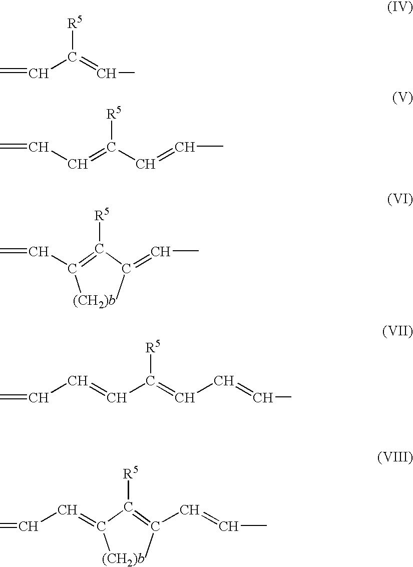
in which R¹ and R², independently of one another, represent a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or can be substituted with 0 to 5 hydroxy groups;
R³ and R⁴, independently of one another, stand for the group —COOE¹, —CONE¹E², —NHCOE¹, —NHCONHE¹, —NE¹E², —OE¹, —OSO₃E¹, —SO₃E¹, —SO₂NHE¹ or -E¹, whereby E¹ and E², independently of one another, represent a hydrogen atom, a C₁-C₄-sulfoalkyl chain, a saturated or unsaturated, branched or straight-chain C₁-C₅₀-alkyl chain, which optionally is interrupted by 0 to 15 oxygen atoms and/or by 0 to 3 carbonyl groups and/or is substituted with 0 to 5 hydroxy groups, R⁵ stands for a hydrogen atom, or a fluorine, chlorine, bromine or iodine atom, Me, Et, or Prop,
b means the number 2 or 3, and
X and Y independently stand for O, S, ═C(CH₃)₂ or —(CH═CH)—, as well as salts and solvates of these compounds.

10. Polynucleotide, especially DNA, RNA or PNA, comprising a sequence that codes for an emitter-binding peptide according to claim 1 or functional variants thereof.

11. DNA- or RNA-vector molecule, which contains at least one or more polynucleotide(s) according to claim 10 and which can be expressed in cells.

12. Host cell that contains a polynucleotide according to claim 10 or a vector molecule according to the invention.

13. Antibodies, especially polyclonal or monoclonal antibodies, human or humanized antibodies, synthetic or recombinant antibodies, comprising at least one emitter-binding peptide according to claim 1.

14. Process for the production of an emitter-binding peptide according to claim 1, comprising the immunization of a suitable organism with an emitter, comprising a dye, which is selected from the group of polymethine dyes, such as dicarbocyanine, tricarbocyanine, indotricarbocyanine, merocyanine, styryl, squarilium and oxonol dyes, and rhodamine dyes, phenoxazine or phenothiazine dyes.

15. Process for the production of an emitter-binding peptide according to claim 1, comprising the recombinant and/or synthetic production of the peptide.

16. Use of an emitter-binding peptide according to claim 1, or a nucleic acid, a host cell, or an antibody, of the invention as a diagnostic agent for in vitro diagnosis.

17. Diagnostic kit for in vitro diagnosis, comprising at least one agent that is selected from an emitter-binding peptide according to claim 1, a nucleic acid, a host cell, or an antibody, of the invention, optionally together with other adjuvants and/or instructions, in common or in separate containers.

18. Process for quantitative in vitro determination of a substance that is contained in a sample, comprising the steps of
a) Bringing into contact an emitter-binding peptide according to claim 1 with an emitter, whereby the interaction of the emitter of the conjugate with the emitter-binding peptide produces a change in the spectral emission properties of the emitter, and
b) Measuring the change in the spectral emission properties of the emitter.

19. Process for direct quantitative in vitro determination of a substance that is contained in a sample or an antigen-detecting agent that is present in the sample according to claim 18, in addition comprising the step of,
d) Quantification of the substance that is contained in the sample by means of the measured change in the emission properties of the emitter.

20. Process according to claim 18, whereby the change in the spectral emission properties of the part of the emitter is selected from a change of the polarization plane, phosphorescence, especially phosphorescence intensity, service life of the fluorescence and a bathochromic shift of the absorption maximum and/or fluorescence maximum.

21. Process according to claim 18, whereby as the emitter-binding peptide, antibody fragments, such as, for example, Fab fragments, scFv fragments, scTCR chains, single-chain-antibodies and mixtures thereof are brought into contact with the sample.

22. Process according to claim 18, whereby the emitter-binding peptide comprises a sequence according to the invention.

23. Process according to claim 19, whereby the emitter-binding peptide has a binding affinity for the emitter of less than 50 nm and preferably less than 10 nm.
